(19) United States
(12) Patent Application Publication
(10) Pub. No.: US 2016/0361014 A1
(43) Pub. Date: Dec. 15, 2016

(54) DEVICES, SYSTEMS AND METHODS FOR TRACKING AND MONITORING ORTHOPAEDIC PATIENTS

(71) Applicant: FOOT INNOVATIONS, LLC, Coatesville, PA (US)

(72) Inventors: Justin M. KANE, Coatesville, PA (US); Vivian WU, Brooklyn, NY (US); Cameron Michael Iroquois FARR, New Paltz, NY (US); Aubrey Elizabeth KALASHIAN, Greenwich, CT (US); Kuniko Carolyn HUNTER, Chester, VT (US); Emily Joanne DIMAMBRO, Gaithersburg, MD (US)

(21) Appl. No.: 15/155,796

(22) Filed: May 16, 2016

Related U.S. Application Data
(60) Provisional application No. 62/161,686, filed on May 14, 2015.

(51) Int. Cl. A61B 5/00; A61B 5/01; A61B 5/11

(52) U.S. Cl. CPC A61B 5/4851; A61B 5/1123; A61B 5/0022; A61B 5/01; A61B 2562/0247

(57) ABSTRACT

A patient monitoring system is provided which includes a patient monitoring device secured to a prosthetic or orthotic. The patient monitoring device includes a spatial orientation sensor, a wireless interface adapted to communicate with a remote wireless device and a processor adapted to detect a wireless device running a software application, establish communications with the detected wireless device using the wireless interface, and transmit data from the spatial orientation sensor to the software application. The data from the spatial orientation sensor can be used to determine the tilt or inclination of the prosthetic or orthotic device from which the elevation of the device can be determined. A method of monitoring a patient wearing a prosthetic or orthotic device is also provided.

This application claims the benefit of Provisional U.S. Patent Application Ser. No. 62/161,686, filed May 14, 2015, pending, which is incorporated by reference herein in its entirety.

### BACKGROUND

Technical Field

This application relates generally to devices, systems and methods for monitoring patients after orthopaedic treatment and, in particular, to devices, systems and methods for monitoring the position of the lower extremity of a patient following orthopaedic treatment of the lower-extremity.

Background of the Technology

Short-term limb immobilization and elevation are critical components of the healing process following many ankle, forefoot, and midfoot surgical procedures [1]. After every invasive lower extremity surgical procedure, surgeons order a post-operative care regimen to assist with patient recovery. These protocols contain specific instructions and schedules for gradual increase of patient activity levels and range of motion to facilitate healing and rehabilitation.

Patients are commonly asked to elevate the leg at least 10 centimeters above heart level. This elevation encourages blood return and prevents blood pooling in the leg, thereby minimizing postoperative complications such as edema, thrombosis, wound breakdown, and pain from swelling [2]. Swelling underneath the cast can cause much of the pain patients experience during recovery, and can lead to complications including compartment syndrome—a condition in which there is increased pressure, usually caused by inflammation, in the body's fascial compartments. This increased pressure can be extremely painful and may also lead to permanent nerve and muscle damage.

Failure to elevate the lower extremity can also cause persistent swelling and skin breakdown, or ankle shifting, altering the fracture after subsidence of swelling [3]. Moving or placing weight on the extremity can cause further damage by rupturing sutures, disrupting the formation of scar tissue, or further aggravating the wounded area, opening portals of entry for pathogens. Any of these complications can disrupt and prolong the healing process.

It is important that the patients follow treatment instructions until they meet with their physician or surgeon for their first postoperative evaluation. Instructions are given as clearly as possible by physicians, but patients may not adhere to these guidelines for a variety of reasons including: a lack of understanding, inconvenience to the patient, discomfort, or forgetfulness. It is not economically feasible for doctors to directly monitor patients to ensure adherence to prescribed postoperative immobilization and elevation even in the immediate postoperative period.

According to the National Center for Health Statistics National Ambulatory Medical Care Survey, there were over ten million physician visits for which ankle, foot and toe musculoskeletal symptoms and complaints were the principal reason for visit in 2010 [4]. This estimate is only a glimpse into the number that occur daily at a global level. Ankle and foot injuries and ailments are not limited to certain geographical locations and affect people of all ages. They can occur as a result of trauma encountered in sports or physical labor, accidental injury, or disease, and as such are not limited to a particular segment of the population. The Hospital for Special Surgery, located in New York, N.Y., reported over 2,000 foot and ankle surgeries in the year of 2012 [5]. The American Hospital Association reports 5,686 registered hospitals in the United States in 2013 [6]. In addition, many nonsurgical conditions, including diabetes, arthritis, venous varicosity, non-surgically reduced fractures and soft tissue injuries, include elevation of the leg as a component of treatment plans. Considering the number of pathologies that can affect the lower limbs, the number of patients requiring some sort of elevation of the leg is enormous.

Reasons why a patient may not comply with a post-operative care regimen are varied. Lack of education, not understanding the instructions or the rationale behind them, language barriers, mental health problems, age limitations, or simply a lack of insight into the necessity of care are all possibilities. Consequently, there is an enormous group of people who are at risk of complications as a result of not following their physician's care recommendations for their condition. If physicians were able to monitor their patients' habits after surgery, they would be able to intervene if care regimens were not followed, and could potentially reduce the need for frequent check-ups after a surgery. In areas where access to medical care is limited or prohibitively expensive, it is even more important to reduce the risk of post-surgical complications; further treatments can be difficult, expensive, or even impossible. While state-of-the-art medical care is common in many areas of the United States and other Westernized countries, it is not as common in many parts of the world, and may even be impossible to come by. Even in facilities like mental hospitals and nursing homes, or in situations where a caretaker is responsible for postoperative care, a device that allows the caretaker real-time monitoring of a patient's activity without constant bedside presence will improve care for the patient and reduce the burden for caretakers.

While the advice to elevate the leg after an injury is widely given by doctors and is generally accepted as effective, there is little research on the quantitative effects of the practice. A device that allows for the tracking of patient habits would additionally offer an opportunity to foster global cooperation for the benefit of mankind. The data that could be collected from such a device would allow researchers to better understand the process of healing after a surgical procedure, which could lead to improved care for patients worldwide.

Reducing lower limb pressure and swelling is crucial to a wide array of medical indications. Long term control of blood flow and fluid retention in the legs is necessary for those with chronic edema, circulatory problems, and cardiovascular disease. Leg swelling reduction techniques and systems are also needed for temporary circumstances, such as pregnant women who may experience lower limb swelling while bedridden during pregnancy.

Leg swelling reduction techniques and systems include: physical positioning and lift systems, which elevate the leg causing gravity to draw fluid out of the leg and reduce venous pooling; and active edema prevention methods and systems, which actively prevent venous pooling using an array of techniques. Systems and method for physical positioning and lift systems and active edema prevention methods are disclosed in [7]-[19].

Patients can be prescribed a postoperative care plan of elevating the leg above heart level as much as possible in the ten day period following the surgery. Goniometers are instruments that may be used to measure angles and can be used to determine elevation. Elevation gauges can also be used to determine elevation. Goniometers and elevation gauges can be used to track and assess both the duration and relative magnitude of elevation. Goniometers and elevation gauges are disclosed in [20]-[24].

There still exists a need for a device that improves communication between a patient and a physician by monitoring the patient's lower extremity positioning and alerting both the physician and the patient when deviation from the postoperative rehabilitation protocol occurs.

### SUMMARY

According to a first embodiment, a system for monitoring a patient wearing a prosthetic or orthotic is provided which comprises:

a patient monitoring device adapted to be secured to the prosthetic or orthotic; and

a remote wireless device running a software application;

wherein the patient monitoring device comprises:
a spatial orientation sensor;
a wireless interface adapted to communicate with the remote wireless device; and
a processor;

wherein the processor is adapted to:
detect the remote wireless device running the software application;
establish communications with the detected remote wireless device using the wireless interface; and
transmit data from the spatial orientation sensor to the software application;

wherein data from the spatial orientation sensor can be used to determine the tilt or inclination of the prosthetic or orthotic device;

wherein the software application is adapted to communicate with the patient monitoring device; and

wherein the software application is adapted to display information regarding the data on a display of the wireless device.

According to a second embodiment, a method of monitoring a patient wearing a prosthetic or orthotic device comprising one or more sensors adapted to determine the spatial orientation of the device and a wireless interface adapted to communicate with a remote wireless device is provided which comprises:

transmitting spatial orientation data from the one or more sensors to the remote wireless device using the wireless interface; and

uploading the spatial orientation data on the wireless device to a computer network;

analyzing the spatial orientation data to determine if the patient is adhering to a predetermined protocol for use of the device; and

forwarding electronic notifications to the patient if the patient is not adhering to the predetermined protocol.

According to a third embodiment, a patient monitoring system is provided which comprises:

a prosthetic or orthotic; and

a patient monitoring device secured to the prosthetic or orthotic, the patient monitoring device comprising:
a spatial orientation sensor;
a wireless interface adapted to communicate with a remote wireless device; and
a processor adapted to detect a wireless device running a software application, establish communications with the detected wireless device using the wireless interface, and transmit data from the spatial orientation sensor to the software application;

wherein data from the spatial orientation sensor can be used to determine the tilt or inclination of the prosthetic or orthotic device.

According to some embodiments, a medical device that is wearable for tracking motion is provided. According to some embodiments, the device will perform one or more of the following functions:

1) Monitor the position of the body part it is attached to in reference to the rest of the body.

2) Monitor the position of the body part it is attached to in reference to a “tare point”.

3) Qualitatively measure where the tracker is over the course of time.

4) Quantitatively measure where the tracker is over the course of time.

5) Store the data with respect to #3 and #4 in a cloud based format.

6) Notify the person wearing the device of the data in #3 and #4 as predefined by a practitioner.

7) Notify the practitioner of all data in #3 and #4

8) Notification will be via blue tooth, WIFI, and via personal smartphone application.

9) Track motion in real time in order to monitor position as it pertains to exercises.

10) Transmit feedback to patient from #9 in order to assure patient is performing said exercises in the predefined manner.

### BRIEF DESCRIPTION OF THE DRAWINGS

The skilled artisan will understand that the drawings, described below, are for illustration purposes only. The drawings are not intended to limit the scope of the present teachings in any way.

FIGS. 1A-1C are schematics showing three designs for the case of a device as described herein wherein FIG. 1A shows a rectangular case having rounded edges, FIG. 1B shows an oval case having rounded edges and FIG. 1C shows a curved, rectangular case having rounded edges.

FIGS. 2A-2D are schematics of a spherical coordinate system in which a 3-axis accelerometer can be used to determine angle or tilt.

FIGS. 3A-3D illustrate an alternative method of determining tilt using an accelerometer in which the angle of each axis of the accelerometer from a reference position is determined individually.

FIG. 4 is a schematic illustration showing the operation of a microfluidic electro-pneumatic sensor which can be used to detect tilt of a patient's lower limb according to some embodiments.

FIG. 5 is a chart showing the components of a microfluidic electro-pneumatic sensor which can be used to measure tilt.

FIG. 6 is a schematic of a Wheatstone bridge circuit which can be used in pneumatic circuits to allow for dynamic pressure feedback and automatic compensation of back pressures, bubbles and environmental pressure changes.

FIG. 7 is a block diagram showing the functionality of software for quantitative and qualitative data analysis.

FIG. 8 is a schematic showing a first embodiment of a phone application for a device as described herein.

FIGS. 9A and 9B are schematics showing additional embodiments of a smart device application for a device as described herein.

FIG. 10 is a schematic showing an additional embodiment of a smart device application for a device as described herein.

FIG. 11 is a schematic showing the device in use wherein the device is secured to a cast on a patient's foot and wherein the foot is shown in a lowered, neutral or level and elevated position.

### DETAILED DESCRIPTION

Devices, systems and methods are described in the exhibits as comprising certain features and/or elements. The description of a device, system or method as comprising certain features or elements in the exhibits does not preclude the incorporation of additional elements and/or features. Devices, systems or methods including a plurality of features or elements are described in the exhibits. According to some embodiments, devices, systems and methods comprising one or more of the recited features and/or elements are provided. According to some embodiments, the elements and/or features of different devices, systems and methods described in the exhibits can be combined. Devices, systems and methods comprising one or more features from each of one or more of the devices, systems and methods described in the exhibits are also provided.

As used herein, a “spatial orientation sensor” is a sensor which can be used to determine spatial orientation (e.g., tilt or inclination with respect to a reference position). Various types of data can be used to determine spatial orientation. According to some embodiments, the sensor is a gyroscope and the data generated by the sensor is angular velocity (angular rate) along one or more rotational axes. According to some embodiments, the sensor is an accelerometer having one or more axes and inclination is determined using the gravity vector and its projection on the one or more axes of the accelerometer. According to some embodiments, the sensor is a microfluidic circuit.

The device as described herein will come into close contact with the user and should therefore operate within a general range of body temperatures. The device should also be resistant to adverse weather conditions. The device can be waterproof to protect against accidental water contact in a shower or rain, for example, or contact with blood or other bodily fluids. According to some embodiments, the device is also able to perform in a temperature range from −20 to 70° C. According to some embodiments, the device is able to withstand an impact force of at least 18 kg (40 lb) in order to prevent breakage as a result of being dropped or from day-to-day wear on the device.

According to some embodiments, the device has a minimum power life of 10 days. The device may be inaccessible to the user while it is in use, so it may not be feasible to recharge the device. According to some embodiments, the device will be able to store at least 32 KB of data, which will allow for the sufficient collection of data for the time the device is being used.

Magnetic resonance imaging (MRI) is a medical imaging technique that is often used to assess fracture apposition in the postoperative recovery process. Patients using this device will not be able to safely undergo MRI treatment.

Sampling rate influences power consumption and overall data set. According to some embodiments, data will be sampled at least once every 5 minutes during use. According to some embodiments, data collection will be limited to a maximum bandwidth of 500 Hz, which is commonly used for small electronic devices. According to some embodiments, data will be measured from at least two degrees of freedom. According to some embodiments, data will be written and read in a possible range of 4-10 bits, as this accommodates the data that will be collected. Output from the device can either be raw data obtained from the various subsystems of the device or processed data from a microprocessor.

According to some embodiments, patients will not be able to access the data storage electronics or the data that the physician is able to see, in order to prevent data falsification. The data can be stored and transmitted in such a way that it cannot be modified after capturing and patients will only have access to read-only files. Patient data that will be stored and transmitted by the device can be associated with a standardized form of identification so that medical confidentiality is maintained between the patient and the physician. According to some embodiments, a unique identifier (e.g., identification number), which only the healthcare provider will have access to, can be used to link a patient with their corresponding device, thereby maintaining anonymity of the patient.

The device can be compatible with iOS, Android, and Windows. Application notifications or text messages can be used to communicate with the user. These types of notifications are widely used by many applications, and will be familiar to the everyday consumer. According to some embodiments, the device will comply with ISO/IEEE 11073 standards for communication between medical devices and external computers [34].

The device should be easy to operate, with little to no user interaction with the actual device. According to some embodiments, the user of the device (either the patient or the caretaker responsible for care) will be able to use the device after less than 30 minutes of instruction from the physician.

According to some embodiments, the device complies with all relevant parts of IEC 60601, which is a series of technical standards for safety and effectiveness of medical electrical equipment [35]. According to some embodiments, the exterior casing does not adversely affect the patient's skin. According to some embodiments, the device is accurate in tracking the elevation of the limb within 2.2 cm (1 inch).

According to some embodiments, the lifetime of the device can average at least three years or roughly 52 usages. This lifetime of the device allows for a total of 3 weeks for the device to be installed on the patient, used by the patient, and sterilized or re-assembled while also accounting for idle time between uses and delays in return are also factored into this estimate.

The device should not cause additional fatigue or discomfort to the patient. Since the average weight of a plaster cast on a leg is 1.36 to 2.27 kilograms, the maximum weight of the device can be less than 0.45 kilograms, which is approximately 20-30% of the weight of the cast.

The width of the device should fit against the user's leg. According to some embodiments, the maximum width of the device is 7.62 cm. The height of the device should fit within the cast, splint, or walking boot. According to some embodiments, the maximum height of the device is 15.24 cm. The depth of the device should fit underneath the cast without disturbing the user. According to some embodiments, the depth or thicken of the device is no larger than 1.9 cm. According to some embodiments, the device will be unobtrusive to 90% of a blind sample.

Since the device will be placed near a potentially open wound and be in close proximity to the skin for a long period of time, infection is a risk that must be minimized. Therefore, the device should be sterile upon usage. To ensure sterility for each usage, the outermost casing of the device can be sterilizable. According to some embodiments, the outermost casing of the device is compliant with either ISO 11135, 11137, or 17665. According to some embodiments, the device is hermetically sealed in a package that is to be opened only when the device is about to be installed. [36] The electronic portion of the device does not need to be sterilized because it is contained in the sterile case, and because sterilization methods like steam or ethylene oxide gas could damage the electronic components.

The main casing should be robust enough to protect and hold the inner electronics for the duration of the product's lifetime, including sterilization. The outermost casing will be close to the skin so it should not react with perspiration, blood, or other bodily fluids, or produce any sort of harmful residue.

The number and frequency of orthopaedic surgeries performed by a practice can vary widely. Consequently, this device may potentially sit idle between usages for weeks or months before it is used. The device should therefore have a shelf life of at least one year without use.

According to some embodiments, the device comprises a sensor, a Bluetooth module, a microprocessor, software and a user interface.

### External Case

The external case must be large enough to enclose the inner electronics of the device. The physical dimensions, shape, ergonomic qualities, and the material used for the external case, however, can be varied to optimize comfort of the user.

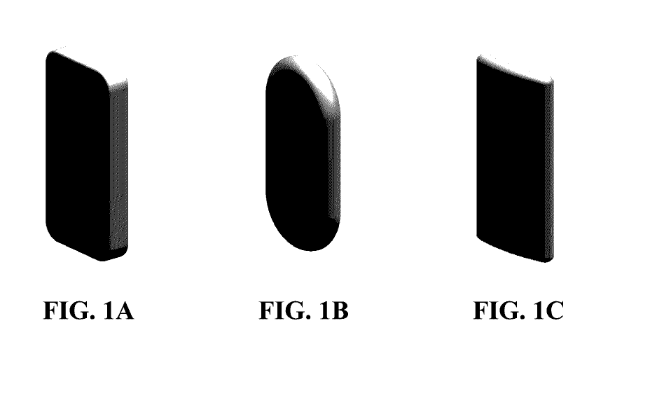

Various case designs are shown in FIG. 1. A rectangular case with rounded edges is shown in FIG. 1A. This case design provides a large amount of area for the electronics but may cause discomfort to the user. An oval shaped case with rounded top and bottom is shown in FIG. 1B. This design would provide more comfort to the user. A rounded, curved rectangular case design is shown in FIG. 1C. This design would be the most comfortable for the user, but would have the least usable volume inside.

A rectangular case with rounded edges, as shown in FIG. 1A, would provide the largest interior volume. This option would be ideal in maximizing space for electronics. However, the larger area of the case would increase the weight of the device. This design would be the least comfortable for the wearer since the flat surface would lie tangent to the wearer's leg, potentially allowing the device to move, further applying pressure to the injured area. The edges and corners of the device, while slightly rounded, may be uncomfortable if pressed into the user's leg. The geometry of this design may also be a challenge for sterilization since sharp corners and edges are especially prone to damage as a result of repeated sterilization.

An oval case with rounded edges is shown in FIG. 1B. A rounded oval shaped case would prevent uncomfortable poking for the user, and would be easier to install comfortably against the wearer's leg. This design would offer slightly less interior space than a rectangular case of equivalent height and width. The rounded edges would also reduce stress and wear on the device since rounded edges are mechanically more favorable than corners.

A rectangular case with rounded edges is shown in FIG. 1C. Since the case is slightly concave, this design would provide the most comfort to the wearer. The curve of the case would allow the device to fit snugly against the wearer's leg, increasing the comfort of wearing the device and also ensuring that the device would stay in place. However, the universality of the device may be compromised since the curved side may not fit comfortably against everyone's leg and since manufacturing a unique casing for each user could be cost-prohibitive. In addition, the curved “C-shape” would reduce the usable space within the device. Moreover, while the volume of a flat rectangular case would be similar, it may be difficult to fit the electronic components around the curved interior.

According to some embodiments, the casing will have dimensions of 7.62×15.24×1.9 cm or smaller. These dimensions are both large enough to house the electronic components of the device, and small enough to comfortably fit against a patient's leg.

Exemplary and non-limiting examples of materials that can be used for the external casing are polycarbonate, acetal, and stainless steel. All of these materials have been used in biomedical devices that have been approved by the U.S. Food and Drug Administration (FDA). While the use of these materials would not guarantee approval, the fact that they have been successfully used in the past indicates that these materials have properties that make them conducive to usage in a biomedical device. Relevant mechanical properties of these three materials are included in Table 1.

TABLE 1
Mechanical properties of polycarbonate, acetal, and stainless steel

| | | Stiffness | Impact strength | |
|---|---|---|---|---|
| | Density (g/cm³) | (GPa) | (J/m) | |
| Polycarbonate | 1.19 [38] | 0.90 [39] | 747.6 | [38] |
| Acetal | 1.41 [40] | 0.85 [39] | 48.06 | [40] |
| Stainless Steel | 8.03 [41] | 200 [42] | 3471.0-5340.0 | [43] |

According to some embodiments, the case of the device can comprise or consist of polycarbonate. Polycarbonate is an amorphous polymer commonly used in electronics such as cell phones and PDAs. As shown in Table 1 above, polycarbonate has a higher impact strength than acetal. Since it is a polymer, it has good electrical insulation properties, which is ideal for protecting the patient from the electronic components within, and it is also fairly resistant to heat. It can be autoclaved, but repeated sterilization can result in surface cracks and decreased fatigue resistance. Polycarbonate also retains its properties when sterilized with radiation or ethylene oxide (EtO). As compared to the other two materials included in Table 1, polycarbonate is cheaper than stainless steel, but is more expensive than acetal [44]. Polycarbonate is also waterproof, which will ensure that the device is resistant to liquids. Polycarbonate is also extremely strong, which will ensure that it is both tamperproof and resistant to drops and day-to-day wear. Polycarbonate will also retain its properties in the temperature range of −20 to 70° C. Polycarbonate is also light. Using the density of polycarbonate (1.19 g/cm³) and the maximum case dimensions (7.62×15.24×1.9 cm), the maximum weight of a polycarbonate case is 0.41 kg. Medical grade polycarbonate is available. While this does not indicate that it will be approved by the FDA for this specific usage, it does indicate that this material has been used successfully in the past. Polycarbonate can be sterilized with EtOH, gamma radiation, or steam. Polycarbonate is not known to be an irritant to human skin, and does not react with bodily fluids or water. It is also a relatively inexpensive material and inexpensive to fabricate.

Acetal or polyacetal resin is a semi-crystalline polymer with high wear resistance, high stiffness, and very good chemical resistance. An exemplary polyacetal resin is sold by E. I. du Pont de Nemours and Company under the trademark Delrin®. As shown in the table above, polyacetal is comparable to polycarbonate in stiffness, but has a much lower impact resistance, because as a semi-crystalline material it is more brittle. The density of polyacetal is also comparable to polycarbonate. Both polymers are much less stiff and dense than stainless steel. Polyacetal cannot be radiation sterilized, but can be autoclaved and EtO sterilized [45].

Stainless steel (e.g., 316L stainless steel) has excellent strength and wear resistance. It is generally corrosion resistant, but repeated steam sterilization can affect its corrosive properties [46]. However, stainless steel is receptive to both radiation and EtO sterilization. Because it is a metal, it has some electrical conductivity, which could potentially present a risk of electrical shock to the patient. It is more expensive than either acetal or polycarbonate, and is heavier per unit volume. However, it is significantly stronger and stiffer, and so it would require a smaller amount of material to achieve the same strength. Therefore, using stainless steel could increase the usable space inside the case by reducing the necessary thickness of the walls. One of the major drawbacks for stainless steel is that since it is a metal, it has the ability to create a Faraday cage effect, preventing the Bluetooth device from communicating. If stainless steel were used, care would needs to be taken to ensure that it does not block the Bluetooth signal. Stainless steel could potentially be used in combination with a polymer to prevent the Faraday cage effect, but a junction between the polymer and steel sections would be structurally weak and a potential contamination risk.

Other materials that can be used for the external case include, but are not limited to, acrylonitrile butadiene styrene (ABS) resin.

### Patient Interface

Once “installed”, the device should not move with respect to the patient's limb for ten days. Any motion of the device with respect to the patient's limb will interfere with the elevation tracking and recording functionality. The device should also be tamper-resistant. While the majority of patients will not maliciously attempt to damage the device, there is always a potential risk of damage to the device. Most crucially, the device should be easy to attach by the physician, and easy to remove after the use period is over. The device should also be compatible with various modalities, including but not limited to plaster casts, ACE-style soft bandages and postoperative surgical boots. Several options to affix the sensor to the patient are described below.

For patients with plaster casts, the device can be placed within the bandages as they are being applied. The physician will apply the sensor when the cast is wet, calibrate the device (e.g., zero the device sensors), and then continue wrapping the plaster cast so that the device is embedded within the cast. Placing the device under the cast will limit patient interaction with the physical sensor. However, this will also limit the size of the device. An advantage is that the device will be completely fixed in place, with the only motion between the sensor and the patient's body arising from micromotion between the cast and the extremity.

The device may be recovered when the cast is cut off of the patient's limb. According to some embodiments, the device's capabilities (e.g., battery life and storage space) exceed the amount of time the cast is in place on the patient. According to some embodiments, the device includes a wireless inductive charging capability to prolong its use cycle.

The sensor may also be applied to the patient's cast or bandages using a non-toxic high strength adhesive. If an adhesive is used, the device should remain in place on the patient until the patient returns to their physician's office. Adhesives such as those used in kinesiology tape and medical cloth tapes can be used. For patients with soft casts or bandages, there is a greater risk of the device moving, as the bandages are elastic and will allow for movement and stretching. In these applications, an adhesive can be used in combination with another connection method. An adhesive can also be used with a plastic postoperative boot. According to some embodiments, the casing of the device can be attached to the boot using either an adhesive or plastic welding.

A third option for patient/device interface is a band or pouch system. This pouch would be outfitted with a lining to firmly hold the device in place (e.g., silicone or rubber), and adjustable bands that would allow the physician to easily install the device on any size leg. If placed in direct contact with the patient's skin, the material should not irritate or cause discomfort to the user. To reduce tampering, the bands may be designed so that when the bands are tampered with an alert is sent in a similar manner to ankle monitors used for house arrest. This type of approach could be implemented to alert the physician if the patient attempts to remove the device. Use of a pouch would allow for simple application and removal of the device by the physician, while keeping it in place during use. It would also allow for quick access should any complications or errors arise with the device. However, this method of attaching the device would increase patient interaction with the device; placing the device outside of the cast would increase the risk of damage or tampering.

The methods of attachment discussed above have been evaluated by using a subsystem decision matrix (Table 2). Key specifications were identified for each technique and relative weights were assigned to each. A score from 1 to 5 was assigned for each of the specifications. Based on the criteria used, the adhesive and wrapped-in-cast appear to be better methods for attachment.

TABLE 2
Decision matrix used to evaluate method to attach the device to the patient

| | | Design Alternative | | |
|---|---|---|---|---|
| | | Band/ | | Wrapped in |
| Specifications | Weight % | Pouch | Adhesive | Cast |
| Overall movement of the | 30% | 2 | 4 | 5 |
| device | | | | |
| after attachment <1 cm | | | | |
| Complies with ISO | 20% | 5 | 3 | 5 |
| 10993 | | | | |
| Can be removed by | 20% | 1 | 3 | 4 |
| physician only | | | | |
| Works with >2 types of | 20% | 5 | 4 | 0 |
| protective wear used | | | | |
| by patients | | | | |
| Can be attached | 10% | 5 | 4 | 3 |
| in <5 minutes | | | | |
| TOTAL | 100% | 3.30 | 3.60 | 3.60 |

Both the adhesive and wrapped in cast attachment methods will ensure that the patient is not able to tamper with the device. These methods also secure the device for the entire duration of the use period, and will prevent the device from moving, which could affect the accuracy of the results. While the wrapped in cast design is effective, this method of attachment cannot be used with protective gear that does not require wrapping. The adhesive method can be used in these applications.

### Electronic Hardware

According to some embodiments, the hardware component of the system comprises a printed circuit board (PCB) which holds a Bluetooth capable microprocessor, and a sensor chip. The sensor chip includes one or more sensors which generate data from which the inclination of the device can be determined. Exemplary sensors include a 3-axis accelerometer, a 3-axis gyroscope or a microfluidic sensor. A battery can also be included to power the system. According to some embodiments, the PCB will also include a slide switch to turn the system on and off and a push button to zero the sensors. According to some embodiments, a small LED light will be included to alert the doctor when the system is ready to be zeroed.

### 2.3.1 Microprocessor

According to some embodiments, the microprocessor has Bluetooth capabilities. According to some embodiments, the microprocessor is a PSoC® 4 with Bluetooth capabilities manufactured by Cypress Semiconductor of San Jose, Calif. The PSoC® 4 microcontroller unit has 128 KB of flash memory to store the program to be executed and up to 16 KB of SRAM where the variable will be stored during the program execution [47]. The PSoC® 4 processor also has a BLE radio (Bluetooth) with a 2.4 GHz transceiver. This is the standard frequency for Bluetooth communication. Bluetooth devices have adaptive frequency hopping to avoid interference between electronic devices. The PSoC® 4 also has both analog to digital converters and digital to analog converters. The processor also has the capabilities to run while in sleep mode to preserve power as part of its power management system. The microcontroller can be programmed prior to being connected to the PCB.

### Gyroscope/Accelerometer Inertial Measurement Unit (IMU)

As set forth above, the device can include a sensor chip which includes a gyroscope or accelerometer. An exemplary, non-limiting example of such a device is the InvenSense MPU-6500 inertial measurement chip manufactured by InvenSense® of San Jose, Calif. This device combines a 3-axis accelerometer and 3-axis gyroscope for 6-axis motion tracking and also includes a Digital Motion Processor™ (DMP). This chip was designed for wearable sensor applications [48]. The chip contains 16-bit analog to digital converters (ADCs), programmable digital filters, a clock, and a temperature sensor. The chip measures 3 mm×3 mm×9 mm. Additional information can be found on the chip's data sheet [49].

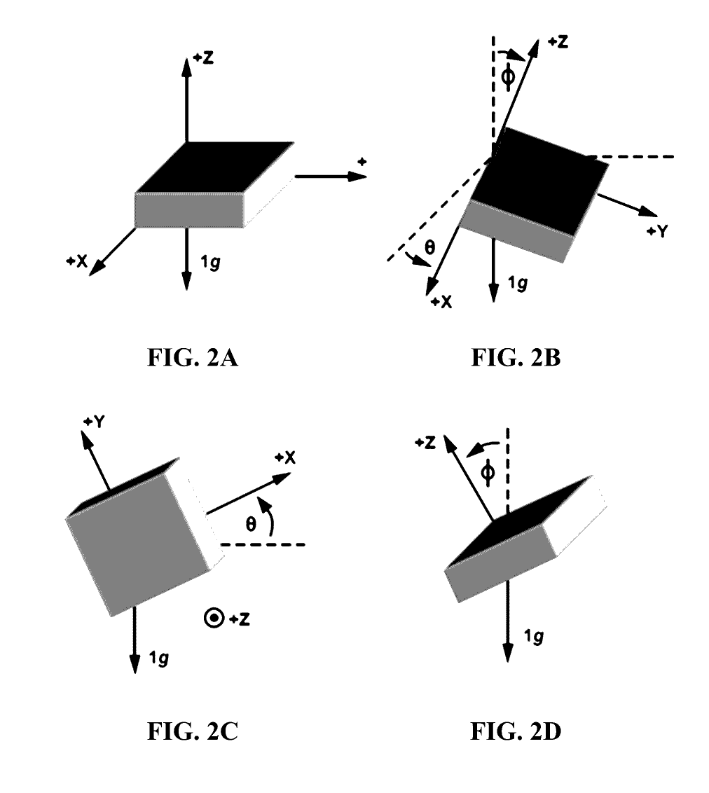

Accelerometers measure both static (due to gravity) and dynamic (due to motion) acceleration [50]. The static acceleration measurements can be used to determine the angle of inclination of the device by using the gravity vector and its projections on each of the axes [51]. As long as no dynamic movements are involved at the time of measurement this method can provide accurate data. FIG. 2 shows the angles of the spherical coordinate system that can be measured using the acceleration data. FIG. 2A shows the orientation of the axes when the device is not rotated. FIGS. 2B-2D show the axes of the device (shown as solid lines) and original axes (shown as dotted lines) when the device is rotated [51]. The spherical coordinates (ρ, θ, φ) can be converted from rectangular coordinates (x, y, z). Theta (θ) is the angle of tilt in the xy plane and phi (φ) is the angle of inclination from the gravity vector. The equations for theta, and phi are as follows:

$$\begin{matrix}θ = {tan}^{- 1} ( \frac{{A}_{x}}{{A}_{y}} ) & eqn . 1 \\ φ = {cos}^{- 1} ( \frac{{A}_{z}}{\sqrt{( {A}_{x}^{2} + {A}_{y}^{2} + {A}_{z}^{2} )}} ) & eqn . 2\end{matrix}$$

Where Aₓ, A_{y}, and A_{z} are the acceleration values in the x, y, and z direction respectively [51]. These measurements are in radians. In order to be converted to degrees they must be multiplied by

$$\frac{180 °}{π} .$$

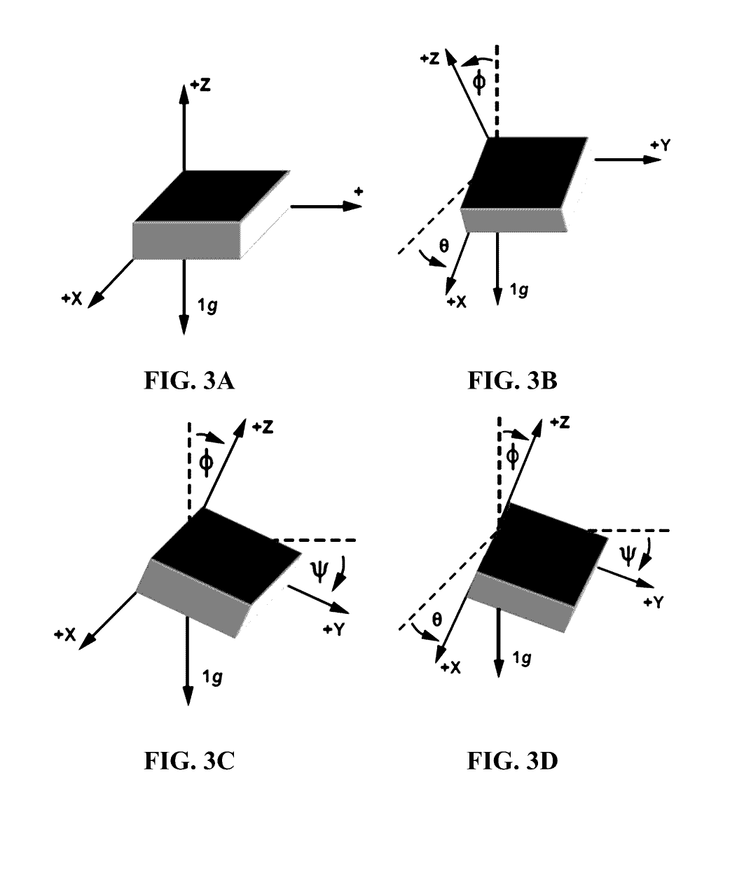

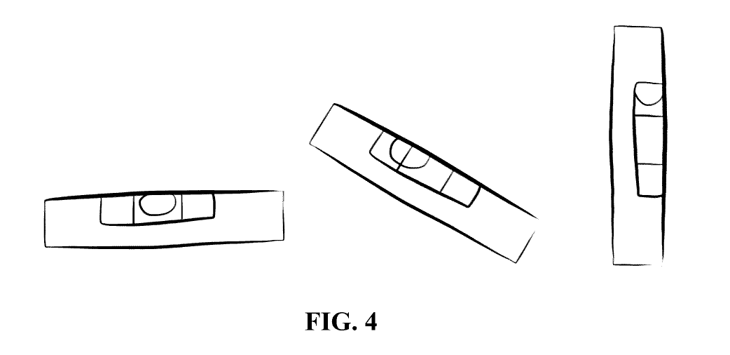

The angle of inclination for each axis can also be determined. The xy plane is considered to be the horizon, and the z axis is perpendicular to the xy plane [51]. The gravity vector points in the negative z direction of the axis system as seen in FIG. 3A. FIGS. 3B-3D show the device tilted in different directions. In FIG. 3, the solid lines represent the axes of the device, and the dotted lines represent the original axes. Theta (θ) is the angle between the xy plane and the x-axis, psi (ψ) is the angle between the xy plane and the y-axis, and phi (φ) is the angle between the gravity vector and the z-axis. The equations for the three angles are:

$$\begin{matrix}θ = {tan}^{- 1} ( \frac{{A}_{x}}{\sqrt{{A}_{y}^{2} + {A}_{z}^{2}}} ) & eqn . 3 \\ ψ = {tan}^{- 1} ( \frac{{A}_{y}}{\sqrt{{A}_{x}^{2} + {A}_{z}^{2}}} ) & eqn . 4 \\ φ = {tan}^{- 1} ( \frac{\sqrt{{A}_{x}^{2} + {A}_{y}^{2}}}{{A}_{z}} ) & eqn . 5\end{matrix}$$

Where Aₓ, A_{y}, and A_{z} are the acceleration values in the x, y, and z direction respectively [51]. These measurements are in radians. In order to be converted to degrees they must be multiplied by

$$\frac{180 °}{π} .$$

By integrating the accelerometer output values twice with respect to time, the position of the device can also be determined.

Gyroscopes measure rotational motion via angular velocity [52]. A 3-axis accelerometer measures the rate of change in the angles around the x, y and z axes [53]. Angle of inclination can be determined from gyroscopes by integrating the output [51]. Accelerometers and gyroscopes measure the same angles in two different manners. Since dynamic motion may be present when the accelerometer is being read, the measurements may not be correct. Measuring angles with a gyroscope, which does not depend on gravity, can allow for more accurate data. According to some embodiments, the outputs from both types of sensors can be compared and averaged to obtain a better value.

The combination of a 3-axis accelerometer and 3-axis gyroscope will allow the angle of inclination and orientation of the lower extremity to be determined. This design allows the movement of the lower extremity to be tracked so the physician would be able to tell if the patient is moving around too much. The system can produce data that can be sent to a smartphone and the physician so that it can be easily interpreted by both parties. The patient will only interact with the GUI, which ensures that the device is easy to use and understand.

This device would allow medically relevant data to be recorded, stored, and used in the future, ensuring that the patient will always receive care tailored to their personal needs. Consumers are already familiar with the concept of wearable electronics; the FitBit and other similar products have exploded on the consumer market. Giving the patient an opportunity to be involved in their own recovery plans will increase adherence to these protocols, and will lead to better health outcomes across the board. Recently, preventative medicine has gained traction as an important part of our healthcare system. Insurance companies are now willing to pay for preventative care, demonstrating that it is an effective means to save money, time, and effort of treating conditions in the future. In countries with socialized medical programs, preventative care is crucial; saving money by reducing expensive complications is in the best interest of the entire system. A recovery tracking device falls under the umbrella of preventative care, as it can help prevent complications from arising as a result of an injury, and can increase patient awareness of their health. In addition, this device will automatically provide documentation for the patient's medical record, affording added protection to both the patient and healthcare provider. If the patient does follow postoperative protocols to poor results, this also removes any doubt of patient non-adherence, and will help the patient to successfully argue for the restitution they deserve.

This design alternative has several potential global impacts. The ability of a doctor to communicate and care for a patient remotely would be extremely valuable in areas where medical care is limited. When patients cannot easily reach a doctor, preventative care becomes even more crucial in ensuring that patients remain as healthy as possible.

This device would reduce the possibility of complications arising in the future, which would greatly reduce the need for follow-up medical care. This device could also potentially reduce the need for or frequency of checkups after surgery. Reducing the number of medical appointments necessary would lift an enormous burden from poor or rural patients who simply don't have the means to see a medical professional. In many developing countries, medical care is limited, and this device would reduce the cost and inconvenience of postoperative care.

This device would also increase the doctor's ability to care for patients who do not speak the same language. This device would ensure that the physician understands the patient's activity after surgery, even if the patient is not able to communicate this information. In many countries, increasing immigration and diversity means that many physicians must treat patients who are not fluent in the same language. This device would help remove this language barrier.

### Microfluidic Electro-pneumatic Sensor

According to some embodiments, the sensor can be a micro-electrofluidic sensor that will detect tilt of the device. From this, the relative elevation of the patient's lower extremity may be derived. As illustrated in FIG. 4, this design takes inspiration from the common carpenter's level, which is used to determine the angle of a surface. The microfluidic electropneumatic sensor is essentially a miniature, automated carpenter's level that detects, quantifies and records the tilt of the patient's limb.

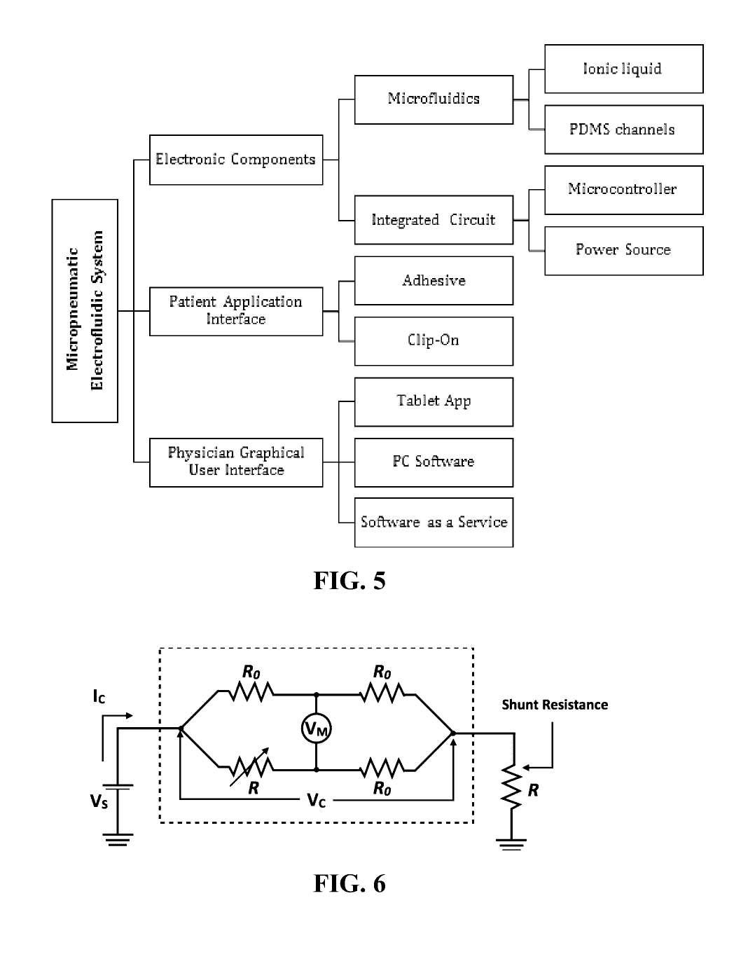

According to some embodiments, the device is comprised of a micro-electro-fluidic circuit, a microcontroller and associated circuitry, patient interface, and external processor and accompanying physician graphical user interface. The subsystem breakdown is shown in FIG. 5. As shown in FIG. 5, the micropneumatic electrofluidic sensor system is comprised of three main subsystems; i) the electronic components; ii) the patient application interface; and iii) the physician's graphical user interface; which are broken down further as shown above.

Electrofluidic circuits are electronic circuits that are made using ionic liquid (IL) filled channels. The sensor can detect relative elevation of the lower leg (i.e. tilt) via tilt-induced electrical resistance variation of the constructed electrofluidic resistor. In use, the sensor can be applied to the patient's leg and zeroed when the patient is in the supine position. When the patient moves from this position, the distribution of IL in the channels will change. Contact with the IL will determine whether or not fluid gates in the walls of the channels are open or closed. The status of the fluid gates will determine the variable resistance of the circuit. This change in voltage will be measured using a microcontroller, and information about the tilt of the leg will be extrapolated from the data.

The microfluidic system can be made from polydimethylsiloxane (PDMS) using multilayer soft lithography (MSL). The sensor will be comprised of a constant electrofluidic resistor and multiple pressure controlled electrofluidic switches. A simplified version of a microfluidic electropneumatic circuit is presented in FIG. 6. FIG. 6 illustrates a Wheatstone bridge circuit that can be used for I-V curve characterization [54].

The microfluidic circuit can be semi-filled with ionic fluid. The level of the fluid in the tube will change with the patient's change in leg positioning. An AC voltage (VS) is applied across the entire circuit. Intermediary switches that open with fluid contact will change the resistance of the circuit. The “gate” is actually a bipolar junction transistor—or a type of transistor that relies on contact with two types of semiconductor to operate [54]. Liquid contact with this junction amplifies the electronic signal. This may be detected in the output voltage (VM) that is measured across the parallel connected electrofluidic switches.

Integrated ionic liquid-based electrofluidic circuits have been demonstrated to have long term stability (with research prototypes showing proper functioning for over 10 days) and temperature stability (up to 100° C.) for pressure sensing in PDMS microfluidic systems [55]. The utilization of Wheatstone bridges in the pneumatic circuits can allow for dynamic pressure feedback and automatic compensation of back pressures, bubbles and environmental pressure changes.

The data recorded by the microcontroller can be processed on an external computer. According to some embodiments, the sensor is physically connected by wire to a computer to transmit data for processing. According to some embodiments, a wireless Bluetooth transmitter is included in the device thereby allowing for immediate, live data transmission and analysis.

There are a number of variations in the design of the sensor that may be made to allow it to better suit its ultimate use. For the liquid that is within the microfluidic circuit, it is possible to use lithium ionic liquids or other ionic fluid electrolytes. Lithium ionic (LI) liquids have improved field-gate performance when used as field gate dielectrics as proposed in this design [56]. LI liquids do have high thermal stability, but due to the use of this device in close proximity to the body, these benefits are not of great use. LI liquids are nonvolatile and compatible with most materials systems.

Ionic liquids may also be used to create bipolar junction transistors. The use of ionic liquids has been demonstrated for the spatiotemporal control of the delivery of biomolecules and ions in biological settings. The devices demonstrating these capabilities were fabricated using standard microfabrication techniques [57]. Changing liquid properties, such as the surface charge and density near the gates, can change the sensitivity of the nanofluidic bipolar transistor [58]. Solid-state polymer electrolytes may also be used and have the following benefits: low volatility at ambient pressure, thermal stability and high ionicity [59]. There are numerous ionic liquids that can be used.

Spacing of gates will determine sensitivity and how robust the constructed circuits are. Inducing a metallic state, for instance, when using ionic liquids containing oxygen, will not have an effect, whereas argon and nitrogen will not have an effect [60]. Field gating is not a simple, linear process; theoretical studies indicate that EDL at the ionic liquid-metal interface involve image changes [61]. Some studies suggest that these type of microfluidic electropneumatic applications could require the development of field doping technologies [61].

Materials and production methodology will assist in determining which ionic liquids to use as well as their relative amounts and concentrations. Graphene and other hydrocarbons have been used to construct ionic-liquid gates [62, 63]. Some studies suggest that these types of microfluidic electropneumatic applications could require the development of field doping technologies or band control to achieve adequate charge doping, energy storage and power supply properties [61, 62].

Devices including a microfluidic sensor have a simple patient interface requiring no patient interaction. It is also difficult to tamper with the internal circuitry of the device. However, it may be difficult to ensure that patients wear the sensor for ten days at a time. Because this device is intended to address the issue of patient adherence to care instructions, it should not be assumed that a patient would be willing to adhere to another instruction to wear a sensor. Discomfort, skin irritation, and sensor loosening are all potential problems with this design alternative. It may be possible for the sensor to be detached from the adhesive portion and then reapplied and reset using a new adhesive at home if the patient desires. This may alleviate the problem of sensor loosening, but it would be difficult to guarantee that the sensor is positioned properly.

Microfluidic circuits present a huge opportunity for expanding technology use in developing nations. Because they are small, inexpensive, and versatile, they may be applied to a wide variety of problems, and are more accessible than traditional electronic sensors.

### Software

The device includes software that will allow the device to operate. The software can be programmed onto the microprocessor prior to the microprocessor being soldered to the PCB. When the device is turned on, it can run an initialization sequence that will include calibration of the sensors. According to some embodiments, the device includes an LED which is lit when the sensors are ready to be zeroed. The device can also include a switch (e.g., a pushbutton) for zeroing the sensors. After the pushbutton has been pressed to zero the sensors, the device can begin recording data. The program can determine how often the sensors are read and when the data will be transferred to a smart device via Bluetooth. According to some embodiments, the program can also convert the raw data into inclination angles and positioning data. According to some embodiments, the program can place the device into a sleep/standby mode during long periods of inactivity, such as when the patient is sleeping, to preserve power.

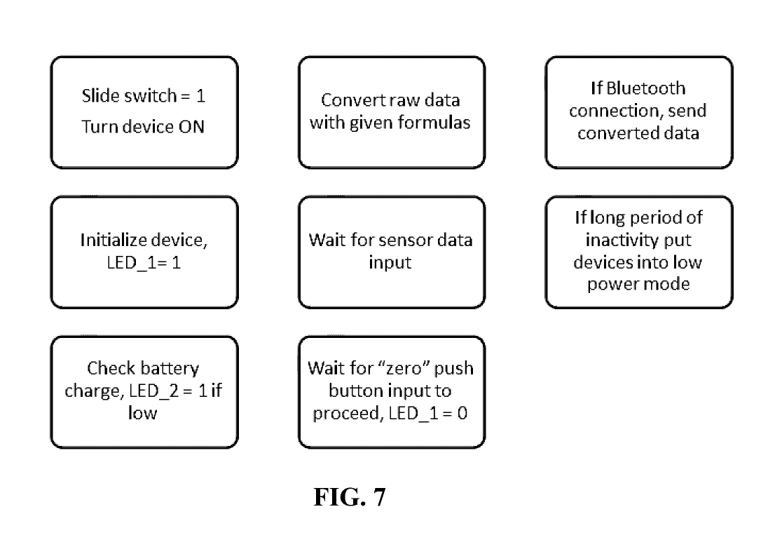

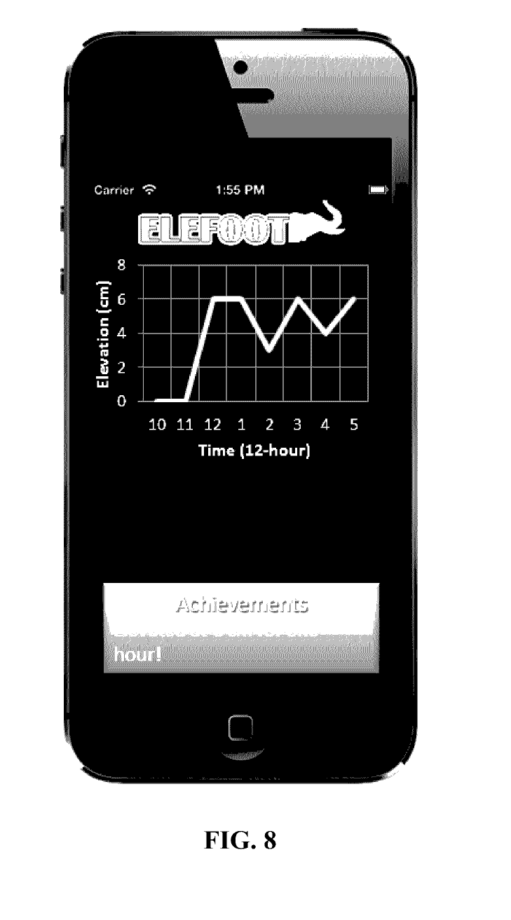

The software's functionality is further illustrated in FIG. 7 which is a block diagram for the proposed functionality of software for quantitative and qualitative data analysis. As shown in FIG. 7, upon the detection of the slide switch in the “on” position, LED_1 will indicate the device has been initialized. Upon initialization, LED_2 will indicate the device has checked the battery charge to ensure the device will last for at least 10 days. LED_3 will indicate a recognized input from the physician to “zero” the device (i.e. to establish the reference position). Once the sensors begin to measure, the program will convert the raw data to elevation data. According to some embodiments, if the elevation is below the “zero” value, a notification will be sent via smart device. If a Bluetooth connection is established, elevation data can also be sent to display progress.

### Graphical User Interface—Smart Phone Application

A smart device application can be used with the device. The application will allow for the user to stay on track during recover by displaying elevation data during the time the monitor is used. Elevation can be displayed real-time on a graph through data transmitted by the monitor through an established Bluetooth connection. If Bluetooth connectivity is lost, the data can be automatically transferred to the application once the connection is regained. The application can allow users to swipe left or right to view past progress. The application can show various types of information regarding the data including, but not limited to, the amount of time the foot has been elevated, elevation heights, and warning/achievement messages.

Warning notifications or achievement messages can be displayed by the application to remind or encourage the user to elevate his or her leg. Notifications can also be sent if the device has been disconnected from the application for an extended period to time, or if the foot has not been elevated for a while. Achievement messages can be sent when the patient is adhering to protocol. The application can also push the collected data to a cloud when the smart device is connected to the internet via wireless so that the physician can also access the information. FIG. 8 is a representation of what the application may look like on a phone. As shown in FIG. 8, the application displays the elevation of the device in centimeters as a function of time for a 12 hour period.

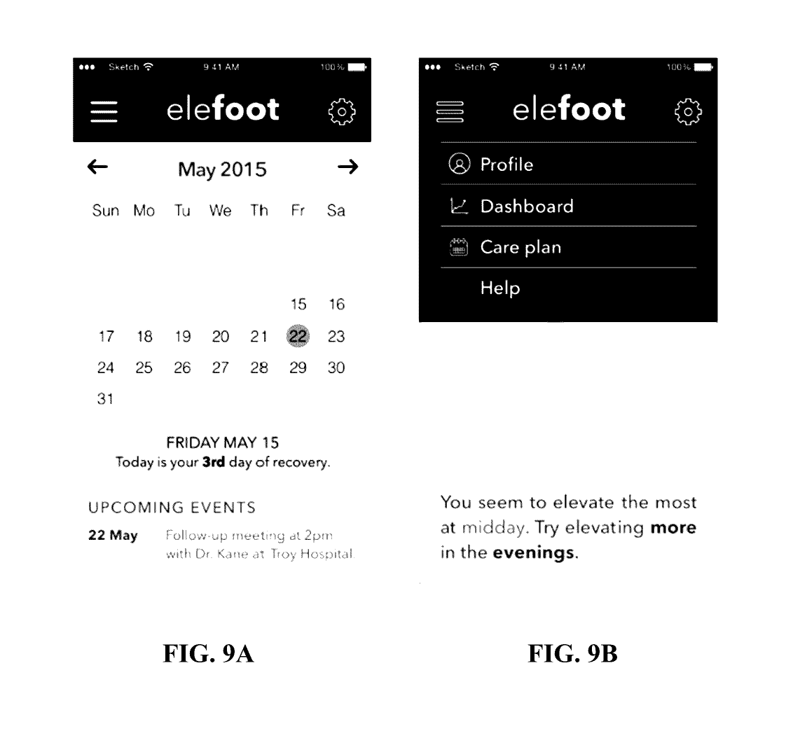

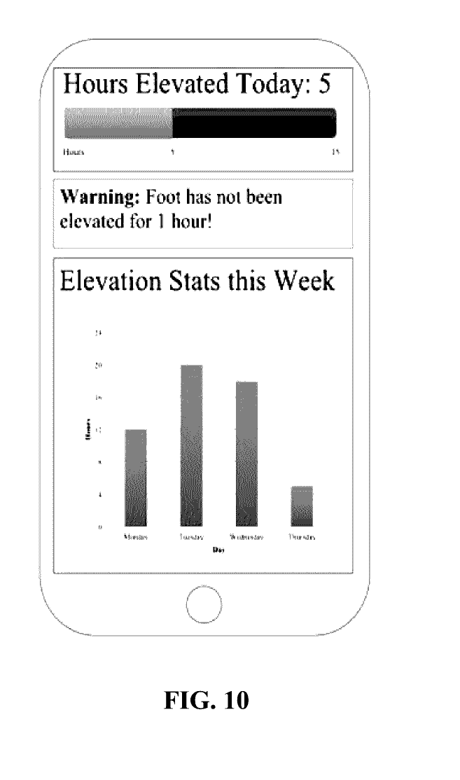

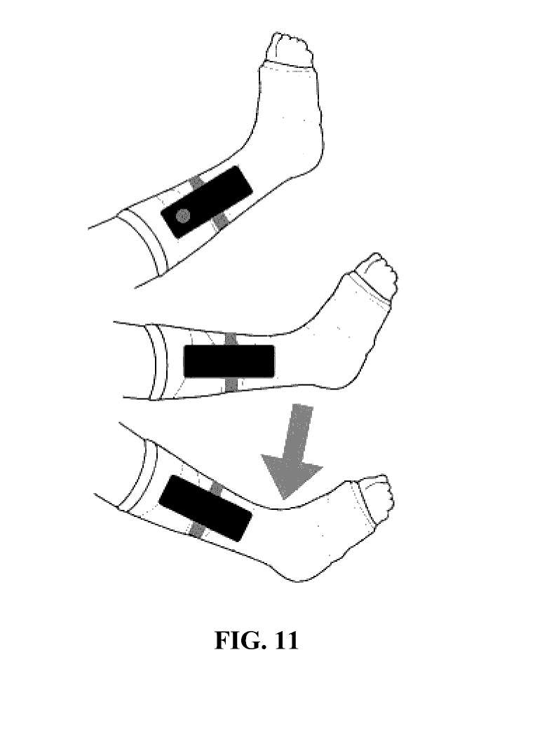

Additional representations of a smart device application are shown in FIGS. 9 and 10. As shown in FIG. 9A, the application can include a calendar function which can include appointment reminders and other information concerning treatment. As shown in FIG. 9B, the application can display how long the device is elevated at various times during the day and can provide suggestions for improving treatment. As shown in FIG. 10, the application can display the number of hours the device is elevated each day and can display a warning if certain goals are not met.

FIG. 11 is a schematic showing the device in use. As shown in FIG. 11, the device is secured to a cast on a patient's lower extremity. FIG. 11 shows the device when the lower extremity is in three different positions: a lowered or un-elevated position (lower schematic); a neutral or level position (middle schematic); and an elevated position (upper schematic).

According to some embodiments, the elevation of the device with respect to the heart is measured. According to some embodiments, the application allows for the patient to select their position (e.g., lying down, sitting up). While this would involve more patient interaction, and the patients would have to change this setting every time they moved, it would allow elevation to be measured with respect to the heart. The application can be programmed such that the application would know the angle of inclination necessary for the foot to be above the heart for different patient positions.

A simple dummy model of the lower extremity (made, for example, out of wood) can be used for testing the device. Using this model, the device can be tested to ensure that the device accurately measures elevation and properly reports the data. The angle of the model leg can be measured using a tape measure and protractor. The reported data from the device can be compared to these actual measurements in order to verify the accuracy of the device.

The device can be tested using accepted testing standards. According to some embodiments, the device can be tested using ISO 10993—Biological evaluation of medical devices, including Part 1: Evaluation and Testing within a risk management process [64]. This standard, set by the International Standards Organization (ISO), is meant to form a framework for testing medical devices. This document categorizes devices based on the nature and duration of contact with the body, and then sets standards to assess the biological safety of the device. The inertial measurement device is a monitoring device therefore classifying it as a medical device [65] and the guidelines set forth in this document will be followed when testing the device to ensure that it is not harmful to the patient.

According to some embodiments, the device complies with UL60601-1 Medical Electrical Equipment, Part 1: General Requirements for Safety which sets forth standards that must be met for medical electrical equipment [66]. These standards include requirements for voltage, current, insulation, and mechanical safety. These standards also include requirements for testing the device to ensure proper operation along with accuracy of output data to prevent hazardous output as well as specifications for labeling.

The case design can be optimized for fabrication. The case can be made using common plastic forming techniques including, but not limited to, injection and compression molding. Both of these forming methods are compatible with acrylonitrile butadiene styrene (ABS) and polycarbonate polymers.

Packaging for the device is also provided. The packaging for the device does not need to be sterile. The packaging can provide protection for the device and appropriate branding and labeling. The packaging can protect the device from damage during shipping and handling. The packaging can also protect the electronic components from humidity to prevent corrosion. According to some embodiments, the packaging conforms to ISO 15223 which identifies the requirements for symbols used in medical device labeling [37].

While the foregoing specification and the attached exhibits teach the principles of the present invention, with examples provided for the purpose of illustration, it will be appreciated by one skilled in the art from reading this disclosure that various changes in form and detail can be made without departing from the true scope of the invention.

### REFERENCES

1. Facaros Z, Kissel S P, Palladino M G, Zgonis T. Postoperative Complications in Foot and Ankle Reconstruction. Perioperative Nursing Clinics, 2011; 6(1): 75-88.
2. Attar F, Selvan D, Machin D, Shariff R, Geary N P. Perioperative Changes in the Microcirculation in Feet After Foot and Ankle Surgery. The Journal of Foot and Ankle Surgery, 2007; 46(4): 238-241.
3. Baraza N, Lever S, Dhukaram V. Home therapy pathway —Safe and streamlined method of initial management of ankle fractures. Foot and Ankle Surgery, 2013; 19(4): 250-254.
4. National Ambulatory Medical Care Survey, 2010; National Center for Health Statistics.
5. Deland J, Roberts M, Foot and Ankle Services. From Department of Orthopaedic Surgery A Return to Form and Function: The Essential Work of Hospital for Special Surgery. New York: Education and Academic Affairs of Hospital of Special Surgery. 2012-2013; 16
6. http://www.aha.org/research/rc/stat-studies/fast-facts.shtml accessed Apr. 6, 2015.
7. Leqin G, Xinhua Z, Guohua M A, Hongyan Q, Jinxia W, Xiaoyi S, Sujuan Z, inventors; Shenzhen Pingle Orthpaedic Hospital, assignee. Lower limb body position pad. State Intellectual Property Office of the People's Republic of China patent CN203379304. 2014 Jan. 8.
8. Law R J, inventor; Rice J, R J Law, assignee. Heel elevation device. Australian Patent Office AU2012216580 (A1). 2013 Apr. 4.
9. Foggio J, Moise V. Therapeutic foot/leg/knee elevation. United States patent US2006272893 (A1). 2006 Dec. 7.
10. Jackson J C. Inflatable orthopedic pillow. U.S. Pat. No. 6,175,979 (B1). 2001 Jan. 23.
11. Chapman C. Device to elevate foot. United States patent US2009112139 (A1). 2009 Apr. 30.
12. Jalloh M. An elevation Device. United Kingdom patent GB2513104 (A). 2014 Oct. 22.
13. Matthews K C, Leos A N. Orthotic system and method utilizing hydrostatic compression of soft tissue to unload knee and heel up to 100%. United States patent US2014276303 (A1). 2014 Sep. 18.
14. Fridlender L M, Maksimova T K. Cushion for legs. Russian Federation patent RU2009133007 (A). 2011 Mar. 10.
15. Kane J R, Christensen S A, Hamilton N, Williams S. Methods and apparatus for increasing blood circulation. United States patent US2008021531 (Al). 2008 Jan. 24.
16. Unsworth J D, Hirsh J, Kaplan R T, Czyrny J J. Neuromuscular electrical stimulation of the foot muscles for prevention of deep vein thrombosis and pulmonary embolism. U.S. Pat. No. 6,615,080. 2003 Sep. 2.
17. Terry D. Lower extremity passive muscle manipulation device and method. World Intellectual Property Organization patent WO2005110327 (A2). 2005 Nov. 24.
18. Spann DC. Surgical body positioner. U.S. Pat. No. 4,185,813 (A). 1980 Jan. 29.
19. Cui H. Device for relieving lower extremity edema of patient in obstetrics and gynecology department. State Intellectual Property Office of the People's Republic of China patent CN202589833 (U). 2012 Dec. 12.
20. Wang F, Tan T, Lui D, Wang A, Chen Y, Li T. Medical angle gauge for measuring elevation angle of bed head. State Intellectual Property Office of the People's Republic of China patent CN203687854 (U). 2014 Jul. 2.
21. Vovk Y M, Vovk O Y, Shmarhaliov A O. Articulated goniometer. Ukrainian Intellectual Property Institute patent UA96234 (U). 2012 Apr. 25.
22. Lee J J. A goniometer for body joints. Republic of Korea patent KR101418057 (B1). 2014 Jul. 10.
23. Gaddipati D, Earley, T. Range of motion system and method. World Intellectual Property Organization patent wO2014160451 (A1). 2014 Oct. 2.
24. Harrison S W, Fadely D W. Pressure transducer elevation gauge. U.S. Pat. No. 5,168,633 (A). 1992 Dec. 8.
25. http://www.diva-portal.org/smash/get/diva2:121327/FULLTEXT01.pdf
26. http://www.who.int/medical_devices/collaborations/force/en/
27. http://www.ema.europa.eu/ema/index.jsp?curl=pages/about_us/general/general_content_000235.jsp&mid=
28. P R Newswire. “Global Orthopedic Braces and Support System Market is Expected to Reach $4,918.7 Million by 2019, at a CAGR of 4.4% from 2014 to 2019.” http://www.prnewswire.com/news-releases/global-orthopedic-braces—support-system-market-is-expected-to-reach-49187-million-by-2019-at-a-cagr-of-44-from-2014-to-2019-500681791.html accessed 25 Apr. 2015.
29. P R Newswire. “Global Foot and Ankle Devices Market 2015-2019: Key Vendors are Arthrex, Stryker and Wright Medical.” http://www.prnewswire.com/news-releases/global-foot-and-ankle-devices-market-2015-2019-key-vendors-are-arthrex-stryker-and-wright-medical-300011332.html accessed 25 Apr. 2015.
30. Sensors Online. Electronics and Computers, “Motion Sensor Market Continues to Grow.” http://www.sensorsmag.com/electronics-computers/news/motion-sensor-market-continues-grow-11868 accessed 25 Apr. 2015.
31. BBC Research LLC. “BBC Research Publishes New Report on Global Market for Wearable Computing Devices.” http://www.bccresearch.com/pressroom/ift/global-market-wearable-computing-devices accessed 25 Apr. 2015.
32. Danova, Tony. Business Insider. “The Wearables Report: Growth trends, consumer attitiudes, and why smartwatches will dominate.” http://www.businessinsider.com/the-wearable-computing-market-report-2014-10 accessed 25 Apr. 2015.
33. World Now. “Growth Opportunities in the Global Wearable Devices Market.” http://www.wkrg.com/story/28860569/growth-opportunities-in-the-global-wearable-devices-market accessed 25 Apr. 2015.
34. International Organization for Standardization. ISO/IEEE 11073-20101:2004, Health informatics—Point-of-care medical device communication—Part 20101: Application profiles—Base standard. http://www.iso.org/iso/home/store/catalogue_tc/catalogue_detail.htm?csnumber=36347
35. International Organization for Standardization. IEC 60601-1-11:2015, Medical electrical equipment—Part 1-11: General requirements for basic safety and essential performance—Collateral standard: Requirements for medical electrical equipment and medical electrical systems used in the home healthcare environment. http://www.iso.org/iso/home/store/catalogue_tc/catalogue_detail.htm?csnumber=65529
36. Sterilization Validation Services. ISO 11135: EtO Sterilization for Medical Devices. Life Science Outsourcing, Inc. http://www.lso-inc.com/sterilization-validation-services/iso11135-eto-sterilization.html
37. International Organization for Standardization. ISO 15223-1:2012, Medical devices—Symbols to be used with medical device labels, labelling and information to be supplied—Part 1: General requirements. http://www.iso.org/iso/home/store/catalogue_tc/catalogue_detail.htm?csnumber=50728
38. http://www.boedeker.com/polyc_p.htm accessed Apr. 6, 2015
39. http://www.matweb.com/reference/flexuralstrength.aspx.accessed Apr. 6, 2015
40. http://www.wshampshire.com/pdf/psg_acetal.pdf accessed Apr. 5, 2015
41. http://www.aksteel.com/pdf/markets_products/stainless/austenitic/316_3161_data_sheet.pdf
42. http://www.nitinol.com/wp-content/uploads/2012/01 /Metals-as-Implantable-Materials.pdf accessed Apr. 6, 2015
43. http://www.stal.com.cn/pdffile/31631613173171.pdf accessed Apr. 5, 2015
44. http:ecnmag.com/articles/2013/11/polycarbonate-vs-fiberglass-and-stainless-steel accessed Apr. 5, 2015
45. http://medical/design.com/prototyping/rd-notebook-when-it-comes-steriliation-matching-methods-materials-paramount accepted Apr. 6, 2015
46. http://biomet3i.com/PDF/IFU/P-IFSCSS Stainless%20Steel%20IFU_SECURED.pdf accessed Apr. 6, 2015
47. Cypress perform, “Programmable System-on-Chip (PSoC®)”, PSoC® 4: PSoC 4XX7_BLE Family Datasheet, November 2014 [Revised December 2014].
48. http://www.invensense.com/mems/gyro/sixaxis.html accessed Apr. 25, 2015.
49. InvenSense Inc., “MPU-6500 Product Specification Revision 1.1”, September 2013 [Revised March 2014].
50. http://www.dimensionengineering.com/info/accelerometers accessed Mar. 8, 2015.
51. Fisher C. Using an Accelerometer for Inclination Sensing. Analog Devices Application Note AN-1057.
52. https://learn.sparkfun.com/tutorials/gyroscope/how-a-gyro-works accessed Mar. 8, 2015
53. http://www.starlino.com/imu_guide.html acessed Apr. 26, 215.
54. He X, Tang N, Gao L, Duan J, Zhang Y, Lu F, Xu F, Wang X, Yang X, Ge W, Shen B. Formation of p-n-p junction with ionic liquid gate in graphene. Applied Physics Letters, 2014; 104, 143102. Retrieved from http://scitation.aip.org/content/aip/journal/apl/104/14/10.1063/1.4870656
55. Wu C Y, Wei-Hao L and Tung Y C. Integrated ionic liquid-based electrofluidic circuits for pressure sensing within polydimethylsiloxane microfluidic systems. Lab Chip, 2011; 11, 1740-1746.
56. Misra R, McCarthy M, Hebard A F. Electric field gating with ionic liquids. Applied Physics Letters, 2007; 90.
57. Tybrandt K, Larsson K C, Gabrielsson E, Jager E W H, Richter-Dahlfors A, Berggren M. Addressable delivery systems based on ion bipolar junction transistors. The Electrochemical Society. 217th ECS Meeting, Abstract #788.
58. Daiguji H, Oka Y, Shirono K. Nanofluidic Diode and Bipolar Transistor. Nano Letters, 2005; 5, 11, pp 2274-2280.
59. Passerini S. Ionic Liquid-based Electrolytes for Next Generation Batteries. University of Muenster, Beyond Lithium Ion, 2012.
60. Li M, Han W, Jiang X, Jeong J, Samand M G, Parkin S S P. Suppression of Ionic Liquid Gate-Induced Metallization of SrTiO3 (001) by Oxygen. Nano Letters, 2013; 13, 10, pp 4675-4678.
61. Ye J T, Inoue S, Kobayashi K, Kasahara Y, Yuan H T, Shimotani H, Iwasa Y. Gates using mysterious ‘Ionic Liquids’ to achieve high surface charge density and superconductivity on an atomically flat film. Nature Materials, 2010; 9, 125.
62. Uesugi E, Goto H, Eguchi R, Fujiwara A, Kubozono Y. Electric double-layer capacitance between an ionic liquid and few-layer graphene. Scientific Reports, 2013; 3, 1595.
63. Kaji Y, Ogawa K, Eguchi R, Goto H, Sugawara Y, Kambe T, Akaike K, Gohda S, Fujiwara A, Kubozono Y. Characteristics of conjugated hydrocarbon based thin film transistor with ionic liquid gate dialectric. Organic Electronics, 2011; 12, 12, pp 2076-2083.
64. http://www.iso.org/iso/catalogue_detail.htm?csnumber=44908 accessed Apr. 25, 2015.
65. https://www.iso.org/obp/ui/#iso:std:iso:10993:-1:ed-4:v1:en accessed Apr. 25, 2015.
66. http://ulstandards.ul.com/standard/?id=60601-1_1 accessed Apr. 25, 2015.

What is claimed is:

1. A system for monitoring a patient wearing a prosthetic or orthotic, the system comprising:
a patient monitoring device adapted to be secured to the prosthetic or orthotic; and
a remote wireless device running a software application;
wherein the patient monitoring device comprises:
a spatial orientation sensor;
a wireless interface adapted to communicate with the remote wireless device; and
a processor;
wherein the processor is adapted to:
detect the remote wireless device running the software application;
establish communications with the detected remote wireless device using the wireless interface; and
transmit data from the spatial orientation sensor to the software application;
wherein data from the spatial orientation sensor can be used to determine the tilt or inclination of the prosthetic or orthotic device;
wherein the software application is adapted to communicate with the patient monitoring device; and
wherein the software application is adapted to display information regarding the data on a display of the wireless device.

2. The system of claim 1, wherein data transmitted from the patient monitoring device includes the spatial orientation of the device.

3. The system of claim 1, wherein the processor is adapted to determine the tilt or inclination of the prosthetic or orthotic device from the data from the motion sensor.

4. The system of claim 1, wherein the spatial orientation sensor comprises an accelerometer and/or a gyroscope.

5. The system of claim 1, further comprising a temperature sensor and/or a pressure sensor.

6. The system of claim 1, wherein the processor is adapted to transmit data from the spatial orientation sensor to the software application automatically.

7. The system of claim 1, wherein the software application is adapted to upload the data to a computer network.

8. The system of claim 1, wherein the software application is adapted to determine the amount of time the prosthetic or orthotic device is oriented in a predetermined position.

9. The system of claim 1, wherein the system comprises an accelerometer.

10. The system of claim 9, wherein data from the accelerometer is used to determine the tilt or inclination of the device.

11. The system of claim 1, wherein the processor is adapted to determine the tilt or inclination of the device based on data from the motion sensor.

12. The system of claim 1, wherein the motion or position sensor comprises an electrofluidic circuit.

13. The system of claim 1, wherein the prosthetic or orthotic device comprises an external case enclosing the spatial orientation sensor, the wireless interface and the processor.

14. The system of claim 13, wherein the external case is rectangular in shape and has rounded corners.

15. The system of claim 14, wherein the external case is curved.

16. The system of claim 1, wherein the application allows the patient to select a body position from a plurality of possible body positions and wherein the application estimates the elevation of the device with respect to the patient's heart for the selected body position.

17. The system of claim 1, wherein the possible body positions comprise lying down and sitting.

18. A method of monitoring a patient wearing a prosthetic or orthotic device comprising one or more sensors adapted to determine the spatial orientation of the device and a wireless interface adapted to communicate with a remote wireless device, the method comprising:
transmitting spatial orientation data from the one or more sensors to the remote wireless device using the wireless interface; and
uploading the spatial orientation data on the wireless device to a computer network;
analyzing the spatial orientation data to determine if the patient is adhering to a predetermined protocol for use of the device; and
forwarding electronic notifications to the patient if the patient is not adhering to the predetermined protocol.

19. The method of claim 18, wherein the one or more sensors include an accelerometer and wherein

20. A patient monitoring system comprising:
a prosthetic or orthotic; and
a patient monitoring device secured to the prosthetic or orthotic, the patient monitoring device comprising:
a spatial orientation sensor;
a wireless interface adapted to communicate with a remote wireless device; and
a processor adapted to detect a wireless device running a software application, establish communications with the detected wireless device using the wireless interface, and transmit data from the spatial orientation sensor to the software application;
wherein data from the spatial orientation sensor can be used to determine the tilt or inclination of the prosthetic or orthotic device.

21. The patient monitoring system of claim 20, wherein the prosthetic or orthotic is an orthopaedic cast.

22. The patient monitoring system of claim 21, wherein the patient monitoring device is secured to the orthopaedic cast by embedding the device into the cast when the cast is applied to the patient.

23. The patient monitoring system of claim 20, wherein the patient monitoring device is secured to a surface of the prosthetic or orthotic using an adhesive.

24. The patient monitoring system of claim 20, wherein the patient monitoring device further comprises memory for data storage.

25. The patient monitoring system of claim 20, wherein the processor is adapted to transmit data from the spatial orientation sensor to the software application automatically.

26. The patient monitoring system of claim 20, wherein the patient monitoring device further comprises a battery.

27. The patient monitoring system of claim 20, wherein the processor is adapted to determine the tilt or inclination of the prosthetic or orthotic device from the data from the motion sensor.

28. The patient monitoring system of claim 20, wherein the patient monitoring device further comprises:
a first switch adapted to turn the device on and off; and
a second switch adapted to zero the spatial orientation sensor when activated.

29. The patient monitoring system of claim 20, wherein the second switch is a push button.
